(19) United States
(12) Patent Application Publication
(10) Pub. No.: US 2018/0344297 A1
(43) Pub. Date: Dec. 6, 2018

(54) SALIVA COLLECTION DEVICE

(71) Applicant: CoWin Biosciences Co., Ltd., Taizhou (CN)

(72) Inventors: Chunxiang WANG, Taizhou (CN); Shanghai NIE, Taizhou (CN)

(73) Assignee: CoWin Biosciences Co., Ltd., Taizhou (CN)

(21) Appl. No.: 15/808,920

(22) Filed: Nov. 10, 2017

(30) Foreign Application Priority Data
Nov. 11, 2016 (CN) 201621216211X

(51) Int. Cl. A61B 10/00

(52) U.S. Cl. CPC A61B 10/0051; B01L 3/502; A61B 10/0096

(57) ABSTRACT

The present invention discloses a saliva collection device provided with a collection tank for containing a liquid and a guide needle for guiding the liquid in the collection tank out of the collection tank, and a vacuum tube, wherein the vacuum tube is used to suck the liquid in the collection tank through the guide needle. The saliva collection device can accurately collect the saliva of a patient who can easily control the position of spitting saliva and the saliva volume, and can avoid collecting the foams in the saliva, so that the volume of the sampled saliva is more accurate.

This application claims the benefit of China application number: CN201621216211X filed on Nov. 11, 2016. The content of this document and the entire disclosure of publications, patents, and patent documents mentioned herein are incorporated by reference.

### TECHNICAL FIELD

The present disclosure relates to the technical field of medical device, and in particular to a saliva collection device.

### BACKGROUND

Saliva is a complex mixture, not only comprises a variety of proteins, but also DNA, RNA, fatty acids and a variety of microorganisms. The diagnosis of disease can be carried out by detection of saliva.

However, in the prior art, when the volume of saliva is determined by the amount of saliva spit out by a patient, there is a problem that the patient is difficult to control the saliva volume. There are foams in the saliva, and the patient often treats the foam in the saliva as the saliva, resulting in the measurement of the saliva volume inaccurate. In addition, there is a variable amount of saliva DNA preservation solution remained in the container which is used to contain the saliva DNA preservation solution in a traditional saliva collection method by cutting a seal film.

### SUMMARY

In view of this, the object of the disclosure is to propose a saliva collection device for convenient collection of saliva.

According to the above object, the saliva collection device provided in the disclosure is provided with a collection tank for containing a liquid and a guide needle for guiding out the liquid from the collection tank.

The saliva collection device further comprises a vacuum tube for sucking the liquid in the collection tank via the guide needle.

In some embodiments of the disclosure, the saliva collection device comprises a housing, a guiding groove, a collection tank and a guide needle, wherein a saliva inlet and a cannula port are provided on the top of the housing, the guiding groove is provided below the saliva inlet, the collection tank is arranged below the cannula port, and the guiding groove is inclined and the lowest point thereof is in communication with the collection tank for flowing the saliva into the collection tank.

In some embodiments of the disclosure, the saliva collection device further comprises a hollow member in communication with the cannula port, the guide needle being secured within the hollow member, wherein one end of the guide needle faces toward the cannula port and the other end thereof extends into the collection tank.

In some embodiments of the disclosure, the saliva collection device further comprises a fixation member, wherein the bottom of the hollow member is threadedly connected to the top of the fixation member, the guide needle is secured within the fixation member, and both ends of the guide needle are exposed on both end faces of the fixation member, respectively.

In some embodiments of the disclosure, the saliva collection device further comprises a connecting member, wherein the hollow member is a hollow cylinder, one end of the hollow member being connected to the cannula port and the other end of the hollow member being connected to the connecting member which is protruded out of the hollow member, and the inner surface of the connecting member is provided with an internal thread and the outer surface of the fixation member is provided with an external thread.

In some embodiments of the disclosure, the saliva inlet has a greater distance in length direction than the distance in width direction, and the distance between the guiding groove and the saliva inlet is gradually reduced or increased along the length direction.

In some embodiments of the disclosure, the saliva inlet is elliptical, rectangular or rounded rectangular.

In some embodiments of the disclosure, the collection tank is of U-shaped or V-shaped, and the guide needle is inserted into the lowest point of the collection tank.

In some embodiments of the present disclosure, the saliva collection device further comprises a vacuum tube, one end of which is provided with a rubber stopper, and the guide needle penetrates into the rubber stopper where the vacuum tube is inserted into the hollow member via the cannula port.

The vacuum tube comprises a transparent tube, a rubber stopper and a protective cover, wherein one end of the transparent tube is closed and the other end thereof is open, the open end is sealed by the rubber stopper, and the rubber stopper is covered with the protective cover.

In some embodiments of the disclosure, the end face of the rubber stopper away from the transparent tube is recessed inwardly to form a recess. Optionally, the protective cover can be a plastic protective cover.

In some embodiments of the disclosure, the vacuum tube contains a saliva DNA preservation solution. The vacuum tube ensures a constant ratio of saliva to saliva DNA preservative solution while preventing the user from contamination of saliva, saliva DNA preservative solution and a mixture of saliva and saliva DNA preservative solution.

In some embodiments of the disclosure, the housing is made of a transparent material.

In some embodiments of the disclosure, a scale line is provided on the housing corresponding to the collection tank.

As can be seen from the above, the saliva collection device provided in the disclosure can accurately collect saliva of a patient, and the patient can easily control the position of spitting saliva and the volume of saliva, and avoid collecting the foam in the saliva so that the volume of the sampled saliva is more accurate. The device sucks saliva in the collection tank via the vacuum tube so as to reduce the risk that the user is contaminated by saliva and the risk that the user is contaminated by a mixture of saliva and saliva DNA preservation solution.

### BRIEF DESCRIPTION OF DRAWINGS

in order to further clearly describe the technical solutions of the embodiments of the present disclosure, a brief description will be given to the drawings which are used in the embodiments. Apparently, the drawings described below are merely some examples of the present disclosure.

FIG. 1 is a cross-sectional view of a saliva collection device of an embodiment of the disclosure;

FIG. 2 is a cross-sectional view of a saliva collection device and a vacuum tube of an embodiment of the disclosure;

FIG. 3 is a cross-sectional view of a vacuum tube of an embodiment of the disclosure;

FIG. 4 is a schematic view of a saliva collection device and a vacuum tube of an embodiment of the disclosure.

### DETAILED DESCRIPTION OF ILLUSTRATIVE EMBODIMENTS

In order to further clarify the object, technical solutions and advantages of the present disclosure, a clear, complete and detailed description will be further given to the technical solutions of the embodiments of the present disclosure. It is apparent that the embodiments described herein are merely some but not all examples of the present disclosure. All the other embodiments derived from the embodiments of the present disclosure by an ordinary person skilled in the art fall in the scope of the present disclosure.

The present invention will be described in further detail with reference to the accompanying drawings, and the present invention will be described in more detail with reference to the accompanying drawings.

The saliva collection device in the disclosure is provided with a collection tank for holding a liquid and a guide needle for guiding the liquid in the collection tank out of the collection tank, and the saliva collection device can collect the liquid conveniently. It is to be noted that the liquid can be saliva or other liquid, and the saliva collection device in the disclosure can also be used for collection of other similar liquids.

Further, the disclosure can further comprise a vacuum tube for sucking the liquid in the collection tank via the guide needle. Therefore, when the guide needle penetrates into the vacuum tube, the liquid in the collection tank can be sucked into the vacuum tube by the pressure difference due to the vacuum degree in the vacuum tube. Therefore, the saliva collection device in the disclosure can not only facilitate the accurate collection of saliva, but also reduce the risk that the user is contaminated by saliva.

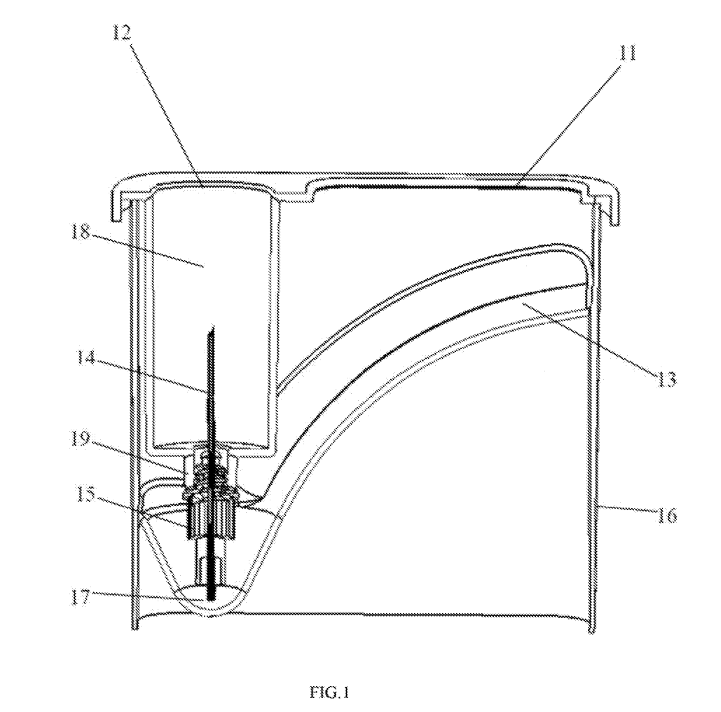

Specifically, referring to FIG. 1, which is a cross-sectional view of a saliva collection device of an embodiment of the disclosure. In the present embodiment, the saliva collection device comprises a housing 16, a guiding groove 13, a collection tank 17 and a guide needle 14, wherein a saliva inlet 11 and a cannula port 12 are arranged on the top of the housing 16, the guiding groove 13 is arranged below the saliva inlet 11, and the collection tank 17 is provided below the cannula port 12, and the guiding groove 13 is in the form of a slope and the lowest point thereof is communicated with the collection tank 17 for flowing the saliva into the collection tank 17. When a patient spits saliva into the saliva inlet 11, the saliva can flow into the collection tank 17 via the guiding groove 13 and is accumulated in the collection tank 17, so that the saliva in the collection tank 17 gradually increases. Further, the saliva collection device further comprises a hollow member 18 in communication with the cannula port 12, which is fixed within the hollow member 18, wherein one end of which is faced toward the cannula port 12 and the other end is inserted into the collection tank 17. As shown in FIG. 1, the end of the guide needle 14 is preferably close to the bottom of the collection tank 17 so that the guide needle 14 can guide the saliva out of the collection tank 17 and avoid the foam in the saliva.

In other embodiments of the disclosure, as shown in FIGS. 1 and 4, the saliva inlet 11 has a greater distance in length direction than that in width direction, so that a patient can spit saliva into the saliva inlet 11 accurately for preventing the saliva from being spit to other places. It is preferable that the distance between the guiding groove 13 and the saliva inlet 11 becomes gradually reduced or gradually increased along the length direction of the saliva inlet 11. As a result, since the distance in the length direction of the saliva inlet 11 is larger, the slope-shaped guiding groove 13 can facilitate the flow of the saliva entered from the saliva inlet 11 into the collection tank 17 to avoid the problems such as inaccurate spitting of saliva and waste of saliva.

In some embodiments of the disclosure, the saliva inlet 11I is elliptical. In other embodiments of the disclosure, the saliva inlet 11 can be rectangular. In still other embodiments of the disclosure, the saliva inlet 11 can be rounded rectangular. These shapes of the saliva inlet 11 can facilitate the patient to align the saliva inlet 11, thereby facilitating the collection of saliva.

In some embodiments of the disclosure, the collection tank 17 is in a form of U-shape, and the guide needle 14 extends into the lowest point of the collection tank 17. In some embodiments of the disclosure, the collection tank 17 can also be in a form of V-shape and the guide needle 14 extends into the lowest point of the collection tank 17. The U-shaped or V-shaped collection tank 17 can facilitate the accumulation of saliva into the collection tank 17 and the introduction of the saliva via the guide needle 14. Nevertheless, since the saliva is extracted from the lowest point of the collection tank 17, it can solve the problem of foams in the extracted saliva, thereby making the volume of saliva more accurate.

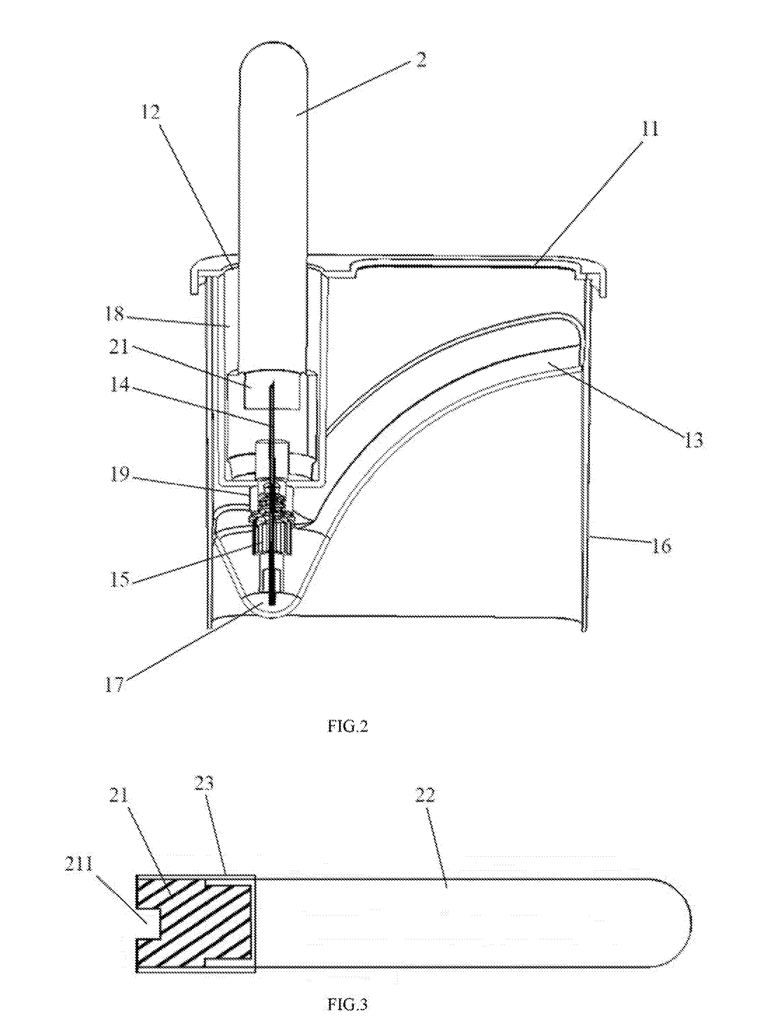

As shown in FIGS. 2-4, in some embodiments of the disclosure, the saliva collection device further comprises a vacuum tube 2, one end of which having a rubber stopper 21, when the vacuum tube 2 is inserted into the hollow member 18, the needle 14 is punctured into the rubber stopper 21. Since the vacuum tube 2 has a degree of vacuum, when the guide needle 14 penetrates into the rubber stopper 21, the saliva in the collection tank 17 flows into the vacuum tube 2 via the guide needle 14. Alternatively, the vacuum tube 2 can be a vacuum saliva DNA preservation tube by which the saliva in the collection tank 17 can be accurately absorbed for accurate sampling. Furthermore, when the vacuum saliva DNA preservation tube is used to contain a saliva DNA preservation solution, all saliva DNA preservation solutions can be quantitatively mixed with saliva. In addition, when the saliva is absorbed by the vacuum saliva DNA preservation tube, the operator does not need to pour the saliva, reducing the risk that the operator is contaminated by the saliva. The use of a vacuum saliva DNA preservation tube with a rubber stopper also eliminates the risk that the operator is contaminated by the saliva DNA preservative solution.

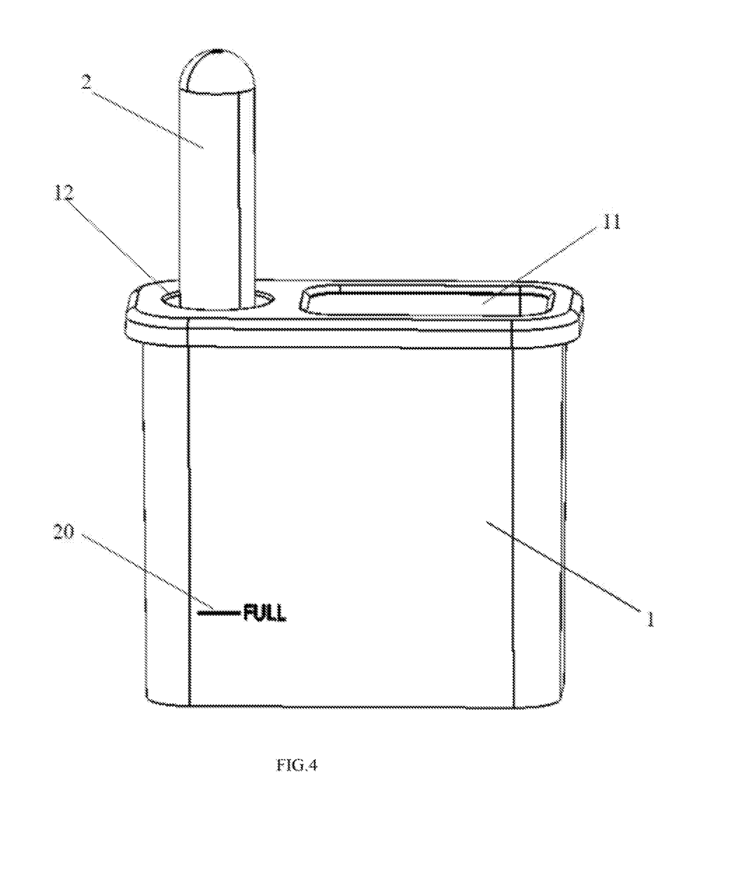

In some embodiments of the invention, as shown in FIG. 3, the vacuum tube 2 comprises a transparent tube 22, a rubber stopper 21 and a protective cover 23, wherein one end of the transparent tube 22 is closed and the other end thereof is open, the open end is sealed by the rubber stopper 21, and the rubber stopper 21 is fitted with the protective cover 23. The protective cover 23 can engage the rubber stopper 21 at the open end of the transparent tube 22, and the rubber stopper 21 can be separated from the transparent tube 22 during centrifugation. Alternatively, the protective cover 23 can be a plastic protective cover. It should be noted that both ends of the protective cover 23 are open, one end being used for engaging the transparent tube 22 and the other end being used for exposing the rubber stopper 21. In a preferred embodiment of the disclosure, the end face of the rubber stopper 21 away from the transparent tube 22 is recessed inwardly to form a recess 211 to prevent the liquid (e.g., saliva, etc.) from contamination of the end face of the rubber stopper 21 when the vacuum tube 2 is pulled out of the guide needle 14.

In some embodiments of the disclosure, the vacuum tube 2 contains a saliva DNA preservation solution. The vacuum tube 2 can ensure a constant ratio of saliva to saliva DNA solution while preventing the user from contamination of saliva, saliva DNA preservative solution and the mixture of saliva and saliva DNA preservation solution.

In some embodiments of the disclosure, as shown in FIG. 1, the saliva collection device further comprises a fixation member IS, wherein the bottom of the hollow member 18 is threadedly connected to the top of the fixation member 15. The guide needle 14 is fixed within the fixation member 15, and both ends of the guide needle 14 are exposed on both end faces of the fixing member 15, respectively. In this embodiment, the guide needle 14 is fixed to the bottom of the hollow member 18 by the fixation member 15, so that the mounting stability of the guide needle 14 in the hollow member 18 can be improved, and the shaking or dislocation of the guide needle 14 at the time of penetrating into the rubber stopper 21 can be prevented.

In some embodiments of the disclosure, the saliva collection device further comprises a connecting member 19 between the hollow member 18 and the fixation member 15, wherein the hollow member can be a hollow cylinder, one end of the hollow member 18 is connected to the cannula port 12 and the other end of the hollow member 18 is connected to the connecting member 19 which is protruded out of the hollow member 18. The inner surface of the connecting member 19 is provided with an internal thread and the outer surface of the fixation member 15 is provided with an external thread. So that, the internal thread on the connecting member 19 and the external thread on the fixation member 15 can be connected by screwing. It is to be noted that the fixation member 15 can be solid so as to securely fix the guide needle 14 in a manner that the fixation member 15 and the guide needle 14 thereon can be easily removed and installed. Preferably, the distance between the end of the guide needle 14 toward the cannula port 12 and the cannula port 12 can be reduced as much as possible so that the rubber stopper 21 on the vacuum tube 2 can be penetrated by the guide needle 14, but the guide needle 14 cannot be exposed on the cannula port 12.

In some embodiments of the disclosure, the housing 16 is made of a transparent material to facilitate observing the volume of saliva accumulated in the collection tank 17 and aligning the guide needle 14 with the rubber stopper 21 of the vacuum tube 2. Preferably, as shown in FIG. 4, a scale line 20 is arranged on the surface of the housing 16 corresponding to the collection tank 17. There can be provided with a plurality of scale lines 20, such as 1 mL, 2 mL, 3 mL, 4 mL, 5 mL, etc., or only two scale lines, such as the minimum scale line and the maximum scale line, so that it can ensure to collect sufficient saliva volume and prevent excess saliva to be collected.

It can be seen that the saliva collection device in the disclosure can accurately collect the saliva of the patient, help the patient to easily control the position of spitting saliva and the volume of the saliva, and avoid collecting the foams in the saliva so that the volume of the sampled saliva is more accurate. The device in the disclosure absorbs saliva from the collection tank via the vacuum tube, so as to reduce the risk that the user is contaminated by the saliva and reduce the risk that the user is contaminated by the mixture of saliva and saliva DNA preservation solution. The saliva collection device has the advantages of simple structure and convenient use, and is convenient for the medical staff to operate so as to improve the working efficiency. In addition, the saliva collection device is low-cost in production and is practical and suitable for application.

It will be understood by those of ordinary skill in the art that the discussion of any of the above embodiments is merely exemplary and is not intended to imply that the scope of the disclosure (including claims) is limited to such examples. In the context of the present invention, the above embodiments or the technical features in the different embodiments may be combined and there are many other variations of the various aspects of the disclosure described above, which are not provided in the details for simplicity. Accordingly, any omissions, modifications, equivalent substitutions, improvements and the like within the spirit and principles of the disclosure are intended to be included within the scope of the present invention.

What is claimed:

1. A saliva collection device, wherein the saliva collection device is provided with a collection tank for containing a liquid and a guide needle for guiding the liquid out of the collection tank.

2. The saliva collection device of claim 1, further comprising a vacuum tube for sucking the liquid in the collection tank via the guide needle.

3. The saliva collection device of claim 1, comprising a housing, a guiding groove, the collection tank and the guide needle, wherein a saliva inlet and a cannula port are provided on the top of the housing, the guiding groove is provided below the saliva inlet, the collection tank is arranged below the cannula port, and the guiding groove is inclined and the lowest point thereof is in communication with the collection tank for flowing the saliva into the collection tank.

4. The saliva collection device of claim 3, further comprising a hollow member in communication with the cannula port, wherein the guide needle is secured within the hollow member, one end of the guide needle facing toward the cannula port and the other end extending into the collection tank.

5. The saliva collection device of claim 1, further comprising a fixation member, wherein the bottom of the hollow member is threadedly connected to the top of the fixation member, the guide needle is fixed within the fixation member, and both ends of the guide needle are exposed on both end faces of the fixation member, respectively.

6. The saliva collection device of claim 5, further comprising a connecting member, wherein the hollow member is a hollow cylinder and one end of the hollow member is connected to the cannula port and the other end of the hollow member is connected to the connecting member which is protruded out of the hollow member, and the inner surface of the connecting member is provided with an internal thread and the outer surface of the fixation member is provided with an external thread.

7. The saliva collection device of claim 3, wherein the saliva inlet has a greater distance in length direction than the distance in width direction, and the distance between the guiding groove and the saliva inlet is gradually reduced or increased along the length direction.

8. The saliva collection device of claim 3, wherein the saliva inlet is elliptical, rectangular or rounded rectangular.

9. The saliva collection device of claim 3, wherein the collection tank is U-shaped or V-shaped, and the guide needle is inserted into the lowest point of the collection tank.

10. The saliva collection device of claim 2, wherein one end of the vacuum tube is provided with a rubber stopper, and the guide needle penetrates into the rubber stopper when the vacuum tube is inserted into the hollow member via the cannula port.

11. The saliva collection device of claim 10, wherein the vacuum tube comprises a transparent tube, the rubber stopper and a protective cover, one end of the transparent tube is closed and the other end is open, the open end of the transparent tube is sealed by the rubber stopper and the rubber stopper is covered with the protective cover.

12. The saliva collection device of claim 11, wherein the end face of the rubber stopper away from the transparent tube is recessed inwardly to form a recess.

13. The saliva collection device of claim 2, wherein the vacuum tube contains a saliva DNA preservation solution.

14. The saliva collection device of claim 13, wherein the housing is made of a transparent material, and a scale line is provided on the housing corresponding to the collection tank.
